(19) United States
(12) Patent Application Publication
(10) Pub. No.: US 2026/0354736 A1
(43) Pub. Date: Nov. 12, 2026

(54) Systems and Methods to Identify Gastric Dysfunction

(71) Applicant: The Board of Trustees of the Leland Stanford Junior University, Stanford, CA (US)

(72) Inventors: Sandya Subramanian, Stanford, CA (US); Todd Coleman, Stanford, CA (US); Linda A. Nguyen, Stanford, CA (US)

(73) Assignee: The Board of Trustees of the Leland Stanford Junior University, Stanford, CA (US)

(21) Appl. No.: 19/123,031

(22) PCT Filed: Oct. 23, 2023
(86) PCT No.: PCT/US2023/077558

§ 371 Date: Apr. 21, 2025

Related U.S. Application Data
(60) Provisional application No. 63/380,436, filed on Oct. 21, 2022.

(51) Int. Cl. A61B 5/392; A61B 5/00; A61B 5/318

(52) U.S. Cl. CPC A61B 5/392; A61B 5/318; A61B 5/4035; A61B 5/4806; A61B 5/7267

(57) ABSTRACT

Embodiments herein provide systems and methods to identify gastric dysfunction. Many embodiments utilize ECG and/or EGG measurements for an individual to determine autonomic modulation and/or gastric motility modulation. Additional embodiments utilize functional principal components analysis to identify features present in ECG and/or EGG that can distinguish phenotypes (e.g., healthy, dysfunctional, etc.) based on scoring features extracted from ECG and/or EGG trace data. Further embodiments provide diagnostic and/or treatments to the individual based on an identified phenotype.

### CROSS-REFERENCE TO RELATED APPLICATIONS

The current application claims the benefit of and priority under 35 U.S.C. § 119(e) to U.S. Provisional Patent Application No. 63/380,436 entitled “Systems and Methods to Identify Gastric Dysfunction” filed Oct. 21, 2022. The disclosure of U.S. Provisional Patent Application No. 63/380,436 is hereby incorporated by reference in its entirety for all purposes.

### STATEMENT REGARDING FEDERALLY SPONSORED RESEARCH OR DEVELOPMENT

This invention was made with Government support under contract FELLOWSHIP NS124835 awarded by the National Institutes of Health. The Government has certain rights in the invention.

### FIELD OF THE INVENTION

The present invention relates generally to myoelectric and electrophysiologic monitoring, more particularly, the invention relates to systems and methods to identify gastrointestinal issues based on neurological monitoring.

### BACKGROUND

The autonomic nervous system is known to be involved in and regulate normal digestive activity in the stomach through both the sympathetic “fight-or-flight” and parasympathetic “rest-and-digest” arms. Specifically, the parasympathetic arm mainly promotes digestive activity through the vagus nerve, while the sympathetic arm restricts digestive activity via the thoracolumbar ganglia. However, this connection between the autonomic nervous system and the stomach can be disrupted or dysfunctional in certain circumstances. Gastroparesis, or delayed emptying of the stomach in the absence of a mechanical obstruction, may be such a circumstance. With a prevalence of 1.5-3%, gastroparesis can occur due to many causes. Two main types of gastropareses are diabetic gastroparesis and idiopathic gastroparesis. Autonomic small-fiber neuropathy is a known part of the progression of diabetes, which may be a contributing factor in diabetic gastroparesis. On the other hand, idiopathic gastroparesis could result in part from autonomic dysfunction, gastric myoelectric dysfunction, or a variety of other contributory causes. Other functional gastrointestinal (GI) diagnoses, such as dumping syndrome (rapid emptying of the stomach) or a physical obstruction preventing digestion, can also involve autonomic dysfunction. Since symptoms can overlap, and symptom resolution does not imply improved gastric emptying, specific sub-typing of gastroparesis into etiology is needed for more targeted interventions.

### SUMMARY OF THE INVENTION

Systems and methods for identifying gastric dysfunction in accordance with embodiments of the invention are illustrated.

One embodiment includes a method for determining a gastrointestinal (GI) phenotype. The method includes obtaining an electrophysiological trace for an individual, extracting a plurality of autonomic features and a plurality of gastric motility features from the electrophysiological trace, generating at least one autonomic dysfunction score based on the extracted plurality of autonomic features, generating at least one gastric dysfunction score based on the extracted plurality of gastric motility features, and determining a GI phenotype based on the extracted autonomic and gastric motility features and the generated autonomic and gastric dysfunction scores.

In a further embodiment, the electrophysiological trace includes electro cardiogram (ECG) and electrogastrogram (EGG) data.

In still another embodiment, the extracted plurality of features comprises instantaneous low frequency power (LF), high frequency power (HF), sympathovagal balance (LF/HF), normalized LF (LFnu), and total autonomic modulation (Totpow).

In a still further embodiment, the phenotype is selected from: healthy, diabetic gastropareses, diabetic non-gastroparesis, and idiopathic gastroparesis.

In yet another embodiment, extracting the plurality of features includes isolating gastric myoelectric information, isolating autonomic information, and identifying sleep and wake times.

In a yet further embodiment, the sleep and wake times are identified using accelerometer data.

In another additional embodiment, the electrophysiological trace is obtained for at least 24 hours.

In a further additional embodiment, the electrophysiological trace is obtained via an ambulatory EGG.

In another embodiment again, the at least one dysfunction score is generated using functional principal components analysis (fPCA).

In a further embodiment again, the extracted plurality of features is used to train a classifier to classify GI phenotypes.

In still yet another embodiment, the classifier comprises multinomial regression, K-nearest neighbor (KNN) with one neighbor, and support vector machine (SVM) with a linear kernel.

In still another additional embodiment, the method further includes generating a treatment plan based on the determined GI phenotype.

In a still further additional embodiment, further including determining an autonomic phenotype based on the extracted plurality of features and the at least one dysfunction score.

One embodiment includes a computer-readable medium comprising instructions which, when executed by a computer, cause the computer to carry out the method for determining a GI phenotype. The method includes obtaining an electrophysiological trace for an individual, extracting a plurality of autonomic features and a plurality of gastric motility features from the electrophysiological trace, generating at least one autonomic dysfunction score based on the extracted plurality of autonomic features, generating at least one gastric dysfunction score based on the extracted plurality of gastric motility features, and determining a GI phenotype based on the extracted autonomic and gastric motility features and the generated autonomic and gastric dysfunction scores.

One embodiment includes a system for determining a GI phenotype, the system includes an accelerometer, an ECG, an EGG, a memory, and a processor comprising a set of one or more processors and a memory containing an identification application, wherein the identification application configures the set of processors to carry out the method for determining a GI phenotype. The method includes obtaining an electrophysiological trace for an individual, extracting a plurality of autonomic features and a plurality of gastric motility features from the electrophysiological trace, generating at least one autonomic dysfunction score based on the extracted plurality of autonomic features, generating at least one gastric dysfunction score based on the extracted plurality of gastric motility features, and determining a GI phenotype based on the extracted autonomic and gastric motility features and the generated autonomic and gastric dysfunction scores.

One embodiment includes a method for determining a GI phenotype. The method includes obtaining an electrophysiological trace for an individual, extracting a plurality of autonomic features and a plurality of gastric motility features from the electrophysiological trace, and determining a GI phenotype based on the extracted autonomic and gastric motility features.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a process of classifying phenotypes of gastroparesis in accordance with an embodiment of the invention.

FIGS. 2A-B illustrate a summary of exemplary autonomic and gastric motility features used for classification, the trends associated with those features, and the custom-defined scores in accordance with an embodiment of the invention.

FIG. 3 illustrates a process to detect a type of gastroparesis using the defined dysfunction scores in accordance with an embodiment of the invention.

FIGS. 4A-B illustrate processed data from two example recordings from a healthy individual and a diabetic gastroparesis patient, respectively, in accordance with an embodiment of the invention.

FIGS. 5A-D illustrate the autonomic activity trends in different individuals in accordance with an embodiment of the invention.

FIG. 6 illustrates box plots of the various dysfunction scores in accordance with an embodiment of the invention.

FIG. 7 illustrates a hardware implementation of the system to identify gastric dysfunction in accordance with an embodiment of the invention.

FIG. 8 illustrates a computing device that can be utilized to perform classification and detection of gastroparesis in accordance with an embodiment of the invention.

FIG. 9 illustrates a network architecture for classifying and detecting gastroparesis in accordance with an embodiment of the invention.

### DETAILED DESCRIPTION

Gastric dysfunction is a medical condition that affects the normal functioning of the stomach. It can cause a range of symptoms, such as pain, bloating, nausea, and vomiting. A common example of gastric dysfunction is gastroparesis. Gastroparesis refers to the paralysis of the stomach, which is a dysfunction that interferes with the nerves and muscles in a human's stomach. Gastroparesis can slow down and/or halt the muscle activity in one's stomach, meaning that any food ingested may sit in the stomach for longer periods of time before being moved to the intestines, even in the absence of any obstruction. Consequently, gastroparesis can lead to the slowing down of a person's digestive process, which is detrimental to their gastrointestinal (GI) functions.

Apart from the GI system, the autonomic nervous system also has a role in regulating human digestive activity. The autonomic nervous system is involved in the regulation of normal digestive activity in the stomach through both its sympathetic and parasympathetic divisions. The sympathetic nervous system controls “fight-or-flight” responses and prepares the body for strenuous physical activity, while the parasympathetic nervous system regulates “rest and digest” functions. Specifically, the parasympathetic nervous system can promote digestive activity through the vagus nerve, while the sympathetic arm restricts digestive activity via the thoracolumbar ganglia. However, this connection between the autonomic nervous system and the stomach can be disrupted or dysfunctional in certain circumstances.

With more than one system contributing to the regulation of digestive activity, the etiology of gastroparesis can be multifactorial. This makes it difficult to determine the root cause of gastroparesis in a patient. In particular, patients with co-morbidities can present complex situations where there are multiple reasons that could have caused and contributed to the gastroparesis. For example, the two main types of gastroparesis are diabetic gastroparesis and idiopathic gastroparesis. While diabetic gastroparesis may appear to suggest diabetes as a reason for gastroparesis, autonomic dysfunctions such as autonomic small-fiber neuropathy, which is a known complication of diabetes, could also be a contributing factor to gastroparesis. On the other hand, idiopathic gastroparesis refers to gastroparesis without a clear and known cause. Idiopathic gastroparesis could be caused by a mix of autonomic dysfunction, gastric myoelectric dysfunction, and a variety of other contributory causes. Other functional GI dysfunctions, such as the dumping syndrome (rapid emptying of the stomach), can also involve autonomic dysfunction. Since different types of gastroparesis may have more than one overlapping cause, the detection and resolution of a single cause may not guarantee improved gastric emptying. It is, therefore, important to be able to disambiguate the different causes, giving rise to a specific classification of various phenotypes of gastroparesis that can result in an etiology that is more accurate and precise.

A challenge to classifying phenotypes of gastroparesis is a lack of multimodal ambulatory monitoring of electrophysiological activity at a high temporal resolution with respect to the autonomic nervous system and digestive activity. In-depth characterization of the gastric-autonomic connection in gastroparesis is necessary to facilitate the accurate classification of various phenotypes of gastroparesis that can lead to better diagnoses. Current attempts at characterizing autonomic and digestive activity in gastroparesis were either not ambulatory, not at a high temporal resolution, not high-quality in terms of physiologically and statistically rigorous methods, or not multimodal. Further, current methods to test for gastroparesis that are approved by the Food and Drug Administration (FDA) and covered by major insurance providers do not provide accurate identification of the phenotype of gastroparesis as a test result, thereby limiting their effectiveness in designing a treatment plan.

Systems and methods in accordance with many embodiments of the invention can improve on current methods by utilizing a two-prong approach that combines recent advancements in ambulatory wearable sensing with modern physiology-based statistical models for heart rate variability and gastric electrophysiology. In many embodiments, systems perform ambulatory monitoring of gastric and autonomic responses of a body having symptoms of gastroparesis and are able to accurately classify the phenotype of the gastroparesis to determine the most likely etiology of the gastroparesis. Systems are able to achieve long-duration remote monitoring of an individual's autonomic and gastric myoelectric activity with an easy-to-implement system compared to current testing methods. In many embodiments, systems include an ambulatory electrocardiogram (aECG) and an ambulatory electrogastrogram (aEGG) configured to record data reflecting the autonomic and gastric myoelectric activity of the wearer. In numerous embodiments, systems utilize ECG and/or EGG measurements for an individual to determine autonomic modulation and/or gastric motility modulation. Sleep is characterized by unique and specific autonomic activity, making it a rich testbed for autonomic regulation. Similarly, the period of digestion after meals can highlight irregularities in digestive activity. This degree of individualized insight can inform clinical management and personalized treatment plans, including type, timing, and dosage of medication, timing of meals and sleep, and the potential therapeutic value of non-pharmacologic solutions such as vagal nerve stimulation. Systems can train a classifier based on recorded data such that the trained classifier can characterize the gastric-autonomic connection of an individual. In some embodiments, systems generate autonomic and gastric dysfunction scores based on recorded data. The generated scores can be used to classify the phenotype of a particular individual's gastroparesis.

In certain embodiments, systems utilize functional principal components analysis to identify features present in ECG and/or EGG that can distinguish phenotypes (e.g., healthy, dysfunctional, etc.) based on scoring features extracted from ECG and/or EGG trace data. Systems can provide diagnostic and/or treatments to the individual based on an identified phenotype. In certain embodiments, systems include a software package that can output the phenotype of gastroparesis of an individual and the most likely etiology of the gastroparesis based on recorded data of the individual and the medical history of the individual. The determinations made by the systems can be displayed on a graphical user interface (GUI) as a part of the software package.

### Classification and Detection of Gastroparesis

In many embodiments, systems measure and record electrophysiological signals of an individual. Systems are able to utilize the recorded measurements to characterize and classify the type of gastroparesis of the individual. Various embodiments utilize a sensor capable of obtaining electrophysiological signals from an individual. Such signals can be obtained by one or more sensors placed on the torso of an individual, including the chest, abdomen, back, thorax, and/or other part of an individual's torso. In various embodiments, the one or more sensors are connected and implemented as a wearable device. In many embodiments, the sensor is an OpenBCI Cyton ambulatory electrophysiological recording system with eight channels placed on the upper abdomen. To better perform ambulatory monitoring, the individual can also manually annotate when they went to sleep and awakened and consumed meals during a 24-hour period of wearing the device.

An example of a wearable sensor is illustrated in U.S. Pat. No. 11,006,838, the disclosure of which is hereby incorporated by reference in its entirety. Wearable devices provide the ability to obtain data outside of a clinical setting to allow for a more holistic view of neuro-gastric health. Such monitoring can include obtaining signals over a period of time (e.g., 4 hours, 6 hours, 8 hours, 12 hours, 16 hours, 18 hours, 24 hours, or more). Such embodiments can include a memory, processor, battery, power source, and/or any other accessory to assist in the operation and/or capture and/or storage of electrophysiological signals. Additional embodiments include an accelerometer to identify the motion and/or physical activity of the individual.

In many embodiments, sensors are placed on the abdomen of an individual, such as to obtain signals for an EGG. As systems also seek to obtain the autonomic function of an individual, sensors can also be utilized to measure the cardiac functions of the wearer. Due to the proximity of an abdomen to the heart, numerous embodiments include sensors capable of obtaining electrocardiac signals in addition to electrogastric signals and/or include additional sensors specific to electrocardiac signals without vastly increasing the size of a wearable device.

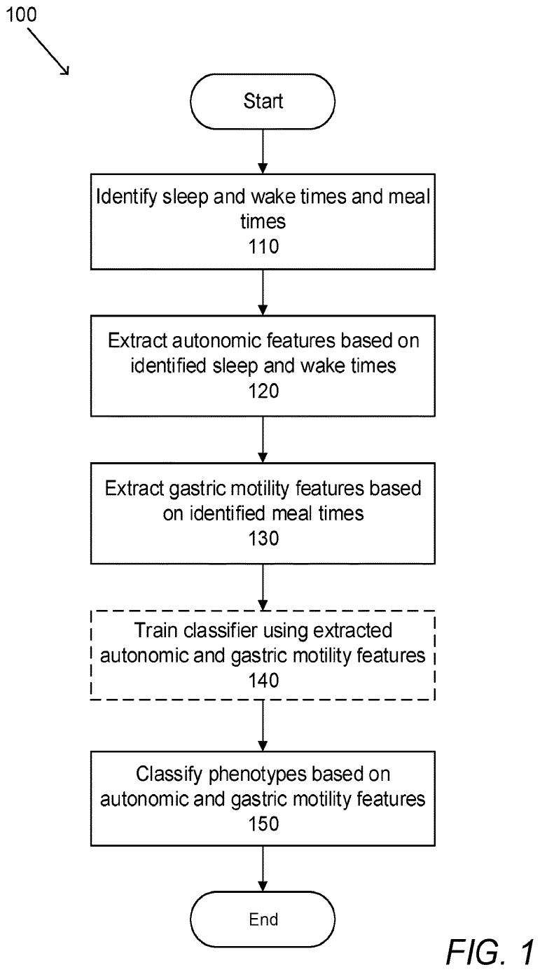

Once the ECG and EEG signals are obtained, systems can characterize and classify the type of gastroparesis. FIG. 1 illustrates a process of classifying phenotypes of gastroparesis in accordance with an embodiment of the invention. In many embodiments, parallel pathways to isolate autonomic features and gastric motility features are utilized. Both sets of information are complementary and non-redundant with respect to physiology, and many embodiments quantify both the intensity of activity and the degree of appropriate regulation of activity. Process 100 identifies (110) sleep and wake times and meal times. Some embodiments obtain annotations of sleep and wake times from an individual being monitored (e.g., patient, subject, etc.). In embodiments including an accelerometer (e.g., a triaxial accelerometer), sleep and wake times can be inferred based on activity. Such methodologies have been described in S. Subramanian, T. P. Coleman (2022). “Automated classification of sleep and wake from single day triaxial accelerometer data,” Proc. 44th IEEE International Conf on Eng in Bio and Med (EMBC); the disclosure of which is hereby incorporated by reference in its entirety. In many embodiments, meal times are identified through manual annotations. Gastric motility features can be extracted based on identified meal times, which will be further discussed below.

Process 100 extracts (120) autonomic features based on identified sleep and wake times. In numerous embodiments, autonomic features are extracted out of the heart rate variability (HRV) of the wearer throughout the day, where the HRV information is collected by an ECG, including an aECG. Extraction of autonomic features from HRV will be further discussed below.

Process 100 extracts (130) gastric motility features based on identified meal times. In many embodiments, gastric myoelectric activity can be extracted from EGG activity in postprandial and fasting periods based on the EGG activity around identified meal times. EGG activity may be obtained from an EGG, including an aEGG.

Process 100 optionally trains (140) a classifier to classify gastroparesis phenotypes using the extracted autonomic and gastric motility features. Training of a classifier may be performed when there is a large set of both autonomic and gastric motility features. Classifiers that can be used to classify phenotypes include but are not limited to multinomial regression, K-nearest neighbor (KNN) with one neighbor, and support vector machine (SVM) with a linear kernel.

Process 100 classifies (150) phenotypes based on autonomic and gastric motility features. For example, gastric motility features can be used to classify GI phenotypes, such as healthy, diabetic gastropareses, diabetic non-gastroparesis, and idiopathic gastroparesis. Autonomic features may be used to classify autonomic phenotypes such as healthy, diabetic, and idiopathic gastroparesis. In some embodiments, the classifier uses a combined set of features to classify phenotypes. The classifier can also operate under a hierarchical classification scheme using the features, where a second-tier classifier is synthesized based on the first-tier classification of the features.

While specific processes for classifying phenotypes of gastroparesis are described above, any of a variety of processes can be utilized to classify phenotypes of gastroparesis as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

Autonomic features are extracted from the measured HRV of the individual. HRV characterizes sympathetic and parasympathetic activity through beat-to-beat differences in the intervals between heartbeats. HRV varies throughout the course of the day based on circadian patterns, internal or external stimuli, and ongoing activity, making it difficult to capture systematic differences within a limited window of measurement. HRV may also vary based on the models used to compute it, especially if such methods are not statistically or physiologically rigorous enough to capture subtle or detailed beat-to-beat differences. HRV information can be identified from ECG using a point process statistical model derived from physiology that yields instantaneous estimates of both time domain and frequency domain HRV measures. Certain embodiments extract times of R peaks from the ECG. In some embodiments, software packages such as the rpeakdetect package in Python 3.0 are used to extract R peak times. Further embodiments compute continuous indices using a point process HRV model.

In many embodiments, systems isolate frequency domain measures, including but not limited to instantaneous low frequency power (LF), high frequency power (HF), sympathovagal balance (LF/HF), normalized LF (LFnu), and total autonomic modulation (Totpow). In general, high frequency power (0.15-0.40 Hz) represents parasympathetic activity, while low frequency power (0.04-0.15 Hz) represents a combination of both sympathetic and parasympathetic activity. Sympathovagal balance is the ratio of LF to HF; total autonomic modulation is the sum of LF and HF; and normalized LF is the proportion of total autonomic modulation that is LF. Many embodiments identify autonomic activity during different periods of the day, including focusing on the overall intensity of autonomic modulation, regulation of circadian patterns, and sleep-wake modulation.

Sleep cycles have an average period of approximately 90 minutes, with some individual variability. To identify sleep cycles from HF, various embodiments filter HF below a certain threshold, for example, below 6e-4 Hz. Using a sliding window, (e.g., 2-hour window, sliding 5 minutes), oscillatory activity can be identified. Various embodiments compute the power of each band. An average power of a band with a period of approximately 90 minutes (e.g., average sleep cycle) can be utilized to identify intensity, and a difference in that average power between sleep and wake can identify appropriate regulation.

Further embodiments analyze the overall modulatory patterns of vagal activity during sleep, irrespective of intensity or magnitude. Many embodiments utilize functional principal components analysis (fPCA) for such identification. (See e.g., J. O. Ramsay, B. W. Silverman (2006). Functional data analysis, 2nd ed. Springer Series in Statistics. New York, NY: Springer; the disclosure of which is hereby incorporated by reference in its entirety.) Various embodiments use vagal activity during overnight sleep from each recording. Such recordings can be filtered (e.g., below 6e-4 Hz). This filtered data can be rescaled (e.g., to be between 0 and 1) and the same duration (x-axis stretching). Certain embodiments then perform fPCA, with the first four functional principal components (fPCs) as well as the associated scores for each recording of sleep. These scores quantify the degree to which the ‘shape’ of the vagal dynamics during each recording of overnight sleep matches the shape of each fPC. For example, a subject's fPC1 score quantifies how similar their vagal sleep pattern is to the first fPC.

In numerous embodiments, gastric myoelectric activity can be captured from the aEGG by computing a spectrogram to determine the power in the frequency band around 0.05 Hz, which is the frequency of the Cajal cells of the human stomach whose action potentials coordinate smooth muscle contractions. Certain embodiments perform artifact rejection and physiologic signal processing methods to extract neuromuscular information from the captured gastric myoelectric activity. Certain embodiments normalize EGG power—for example, the power can be normalized as the mean power in the 0.04-0.06 Hz band minus the background noise level in the 0.06-0.10 Hz band to control for noise variability between recordings and channels. Various embodiments use manual annotations (e.g., from the subject, patient, caretaker, etc.) of mealtimes and content to meals as either meals or snacks. Certain meals can be isolated, such as meals with at least 4 hours of fasting preceding the meal and at least 3 hours of data after the meal. Certain embodiments compute an area under the normalized EGG power curve (AUC) during the 4-hour postprandial period and compare it to the AUC of fasting periods.

In selected embodiments, fPCA analysis can be used to assess the modulation patterns of gastric motility during the postprandial period, irrespective of intensity or magnitude. To do this, certain embodiments rescale the absolute postprandial (4-hour) power around 0.05 Hz after isolated meals between 0 and 1. Since signals should be the same length for fPCA, certain embodiments can rescale the duration of the one isolated meal having slightly less than 4 hours of postprandial data to be the same length as the others. Such embodiments then perform an fPCA analysis, focusing on the first four fPCs, and compute the associated scores.

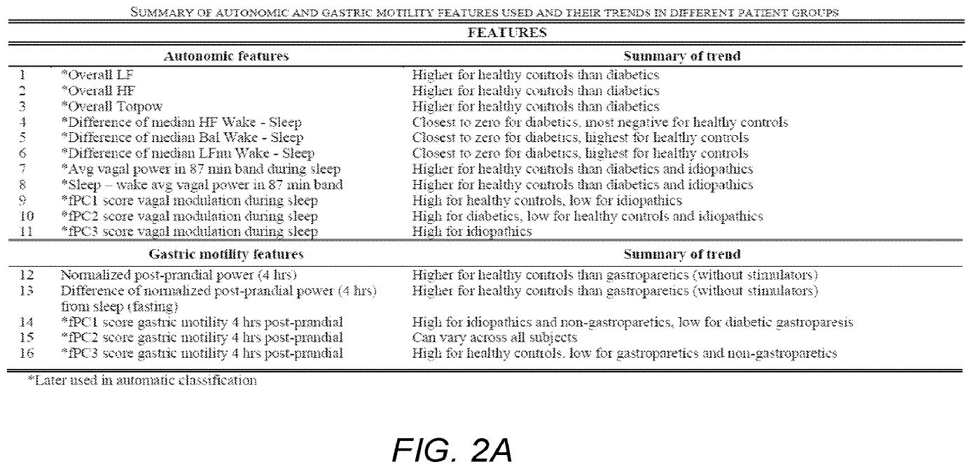

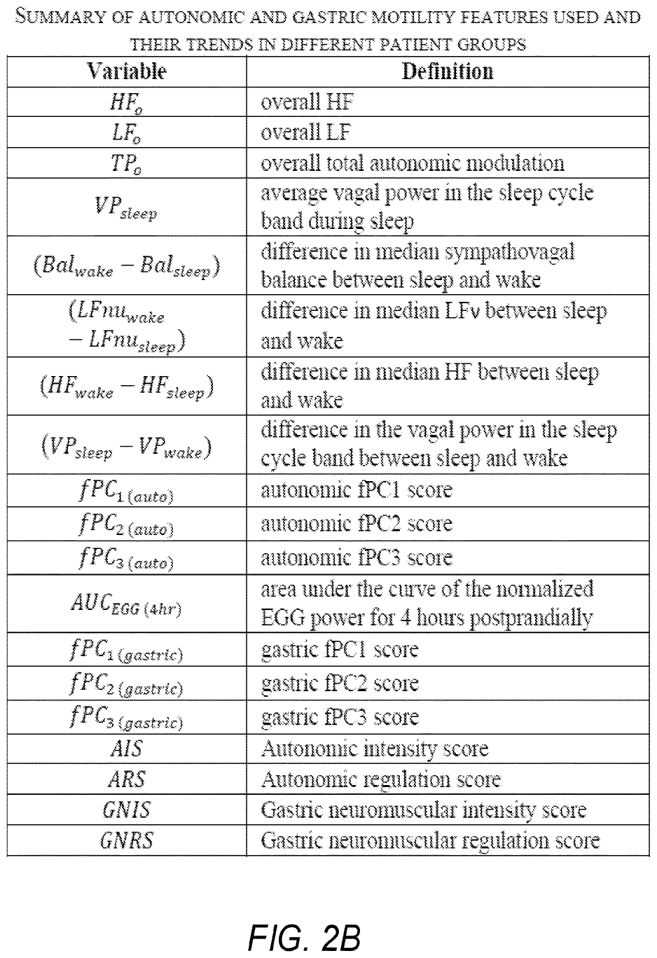

FIG. 2A illustrates exemplary autonomic and gastric motility features used for classification and trends associated with those features in accordance with an embodiment of the invention. For the fPCA scores, certain embodiments project fPC scores on the resulting fPCs of data recordings. In many embodiments, autonomic and gastric motility features can be used to compute autonomic and gastric motility scores. Further embodiments use custom-defined quantitative function scores focusing on the intensity of and the appropriate regulation of activity—exemplary custom-defined scores include an autonomic intensity score (AIS), an autonomic regulation score (ARS), a gastric neuromuscular intensity score (GNIS), and a gastric neuromuscular regulation score (GNRS), where score was defined using features relevant to that score. FIG. 2B illustrates a summary of features, including the custom-defined scores in accordance with an embodiment of the invention. For example, the autonomic intensity score can be defined using the levels of overall autonomic modulation and the intensity of sleep cycles. In contrast, the autonomic regulation score can be defined using the difference in autonomic modulation between sleep and wake, the difference in sleep cycle intensity between sleep and wake, and the qualitative vagal modulatory patterns (fPCA analysis). Autonomic scores can be calculated for each 24-hour recording, while gastric neuromuscular scores can be calculated for each isolated meal. Exemplary definitions of each score are provided below:

AIS=VPₛₗₑₑₚ+HFₒ+LFₒ+TPₒ (1)

ARS=(Bal_{wake}−Balₛₗₑₑₚ)+(LFnu_{wake}−LFnuₛₗₑₑₚ)+(HF_{wake}−HFₛₗₑₑₚ)+(VPₛₗₑₑₚ−VP_{wake})+fPC₁₍ₐᵤₜₒ₎−fPC₂₍ₐᵤₜₒ₎−fPC₃₍ₐᵤₜₒ₎ (2)

GNIS=AUC_{EGG(4hr)} (3)

GNRS=fPC_{2(gastric)}+fPC_{3(gastric)}−fPC_{1(gastric)} (4)

Each of the custom-defined scores can be validated by showing that each was able to distinguish between relevant autonomic and/or GI phenotypes and provided non-redundant insight into the difference between groups.

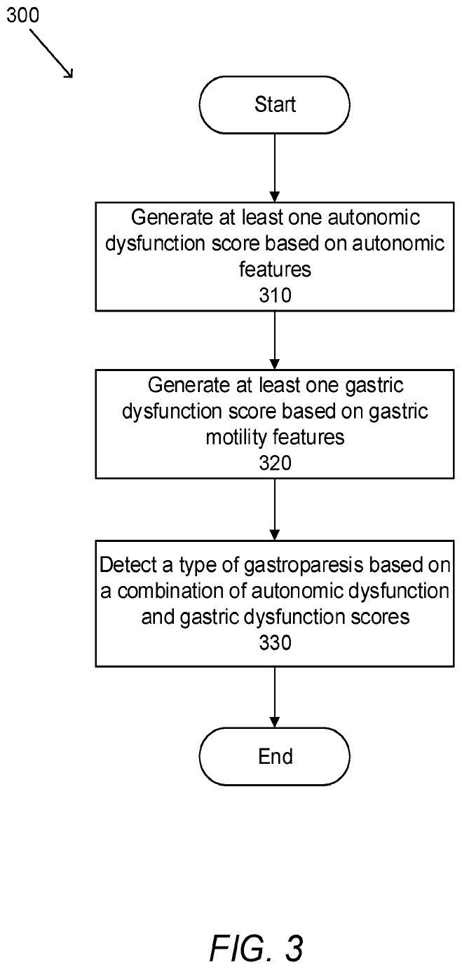

FIG. 3 illustrates a process to detect a type of gastroparesis using the defined dysfunction scores in accordance with an embodiment of the invention. Process 300 generates (310) at least one autonomic dysfunction score based on autonomic features. In numerous embodiments, the at least one autonomic dysfunction score includes but is not limited to AIS and ARS. Process 300 generates (320) at least one gastric dysfunction score based on gastric motility features. In many embodiments, the at least one gastric dysfunction score includes but is not limited to GNIS and GNRS.

Process 300 detects (330) a type of gastroparesis based on a combination of autonomic dysfunction and gastric dysfunction scores. The autonomic dysfunction and gastric dysfunction scores can serve as indicators of the autonomic and gastric conditions of an individual, which can assist with generating a treatment plan. In some embodiments, systems can incorporate the generated scores into a classifier trained to classify phenotypes. Systems can use the combination of the classifier and dysfunction scores to detect the type of gastroparesis of an individual based on the monitored autonomic and gastric myoelectric activity of the individual. In various embodiments, the autonomic and gastric myoelectric activities are obtained from the EGG and/or ECG data from the individual. Such data can be obtained as trace data and/or raw data, while other embodiments obtain autonomic and/or GI motility scores from an individual. Measurements can be obtained over a period of time (e.g., 1-24 hours, such as described herein). Such measurements can be used to identify autonomic and/or gastric motility scores, such as through methods described elsewhere herein, or the scores can be obtained directly from another source (e.g., database or repository). In several embodiments, the detected type of gastroparesis, the generated dysfunction scores, and treatment plans can be displayed on a GUI of an overall system software package. Treatment plans can include pharmacological, pharmaceutical, interventive, behavioral, dietary, rehabilitative, prehabilitative, and/or any relevant treatment as appropriate for the phenotype.

While specific processes for detecting a type of gastroparesis are described above, any of a variety of processes can be utilized to detect gastroparesis as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

### Measurements

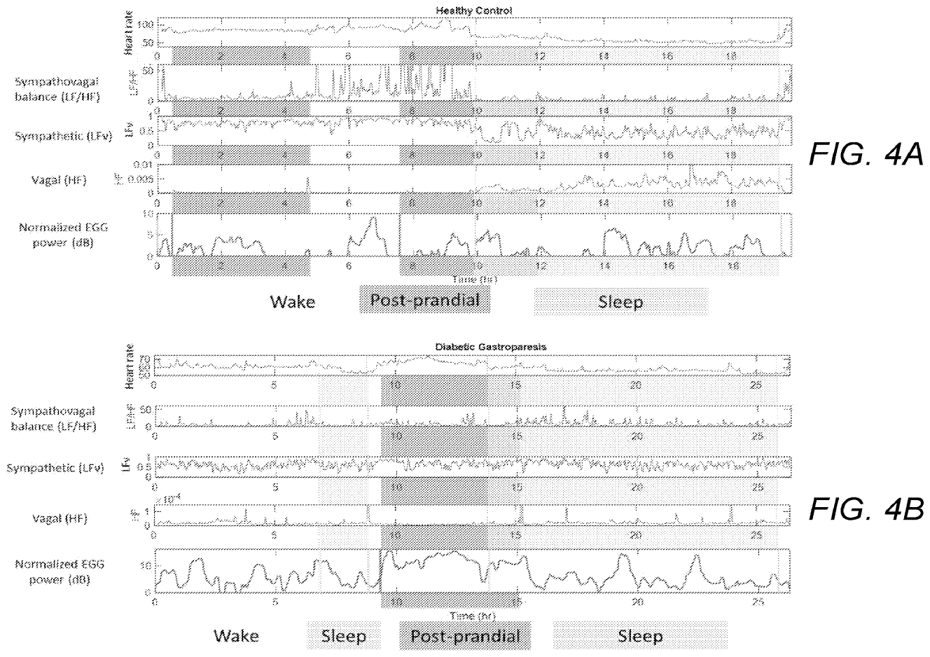

Systems to identify gastroparesis as described herein have been empirically validated via testing. Test results are provided for illustrative and informational purposes. FIGS. 4A-B illustrate processed data from two example recordings from a healthy individual and a diabetic gastroparesis patient, respectively, in accordance with an embodiment of the invention. In each plot, both autonomic and digestive activity markers are shown, including LF/HF, LFv, and HF for autonomic activity, and absolute and normalized power around 0.05 Hz for digestive activity. General periods of wake and sleep, as well as meals, are marked.

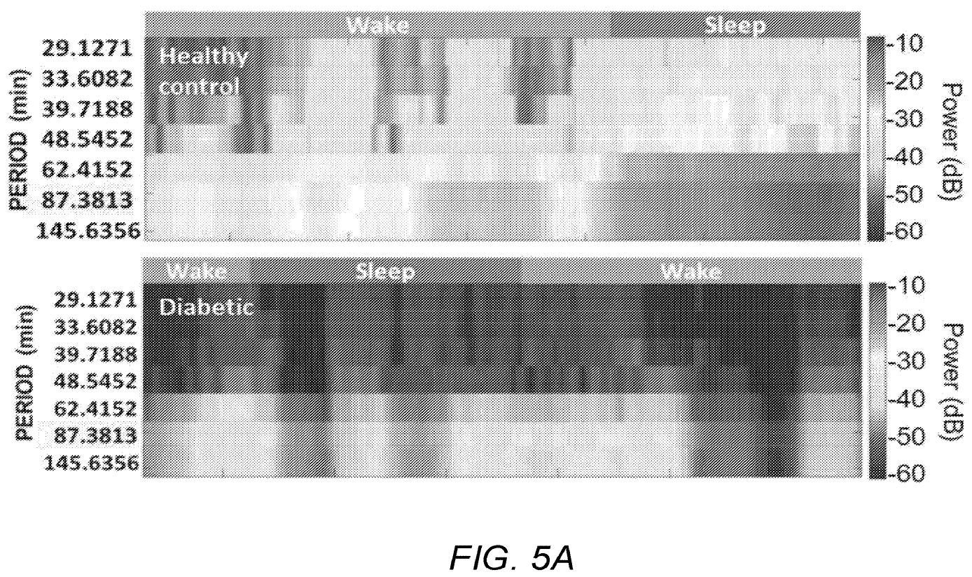

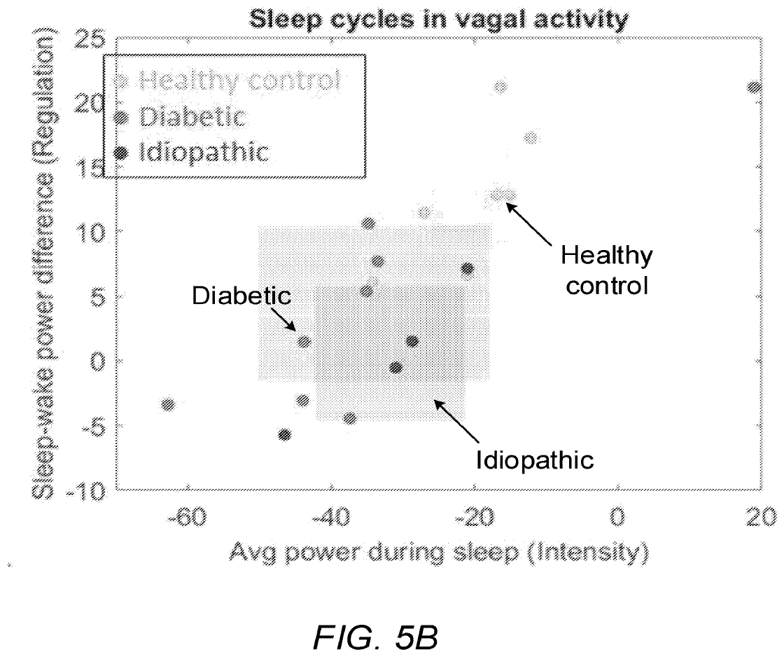

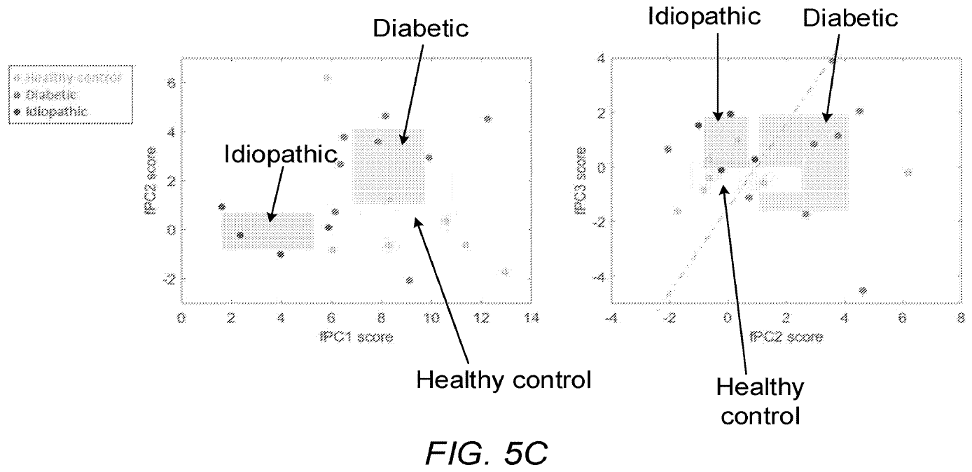

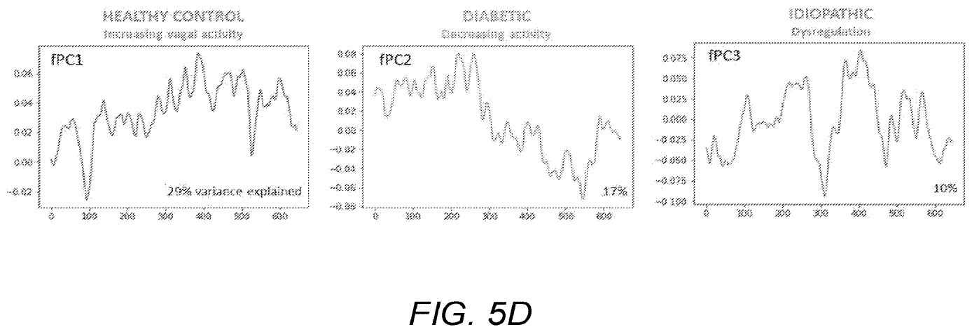

FIGS. 5A-D illustrate the autonomic activity trends in different individuals in accordance with an embodiment of the invention. FIG. 5A illustrates two examples of spectrograms of vagal activity from two different 24-hour recordings, one from a healthy individual and one from a diabetic gastroparesis individual. In both cases, the general periods of wake and sleep are marked. The frequency band of interest, with a period of approximately 87 minutes, is the second from the bottom on both spectrograms. To quantify these differences, FIG. 5B illustrates the comparison of average power in the 87-minute period band during sleep (sleep cycle intensity) and the difference in power in the 87-minute band between sleep and wake (sleep cycle regulation) for all recordings. The greatest power in that band during sleep and the greatest separation between wake and sleep both occur for healthy controls, which span the top right section of the plot. At a group level, both sets of patient groups (diabetics and idiopathic gastroparesis) have decreased power in that band during sleep compared to healthy controls (decreased intensity of activity). Both sets of patient groups also have decreased separation between sleep and wake (impaired regulation). Since this difference is computed by subtracting the power during wake from that during sleep, the healthy controls have a positive difference, indicating that there is a much higher power in that band during sleep than during wake, which agrees with the notion of sleep cycles. Interestingly, the two patient groups seem to separate further from each other based on whether they have greater disruption to the overall intensity of sleep cycles or sleep-wake regulation. For example, the diabetics seem to be higher on the y-axis but more to the left on the x-axis than the idiopathic gastroparesis subjects. This suggests that they are characterized more by decreased intensity of sleep cycles rather than dysregulation. On the other hand, the idiopathic gastroparesis subjects are lower on the y-axis but slightly to the right of the diabetics on the x-axis, suggesting that they are characterized more by dysregulated sleep cycles rather than decreased intensity.

FIG. 5C illustrates the functional principal component (fPC) scores of each of the 24-hour recordings plotted against each other for the first three fPCs. FIG. 5D illustrates the patterns of vagal activity during sleep represented by the first three functional principal components.

The first fPC, which explains 29% of the variance, shows increasing vagal activity throughout the duration of sleep. This fPC has the highest scores for healthy controls. The second fPC, which explains 17% of the variance, shows decreasing vagal activity through the course of sleep and specifically, a decreased intensity of activity for most of the duration of sleep. This fPC has the highest scores for the diabetics. The third fPC, which explains 10% of the variance, shows a variable pattern of vagal activity with several peaks and troughs. While the intensity of activity overall is not markedly reduced, the pattern of activity is highly variable. This fPC has the highest scores for the idiopathic gastroparesis subjects. This difference in vagal dynamics over the course of sleep between the different subgroups, specifically in terms of both intensity and regulatory pattern, may relate to underlying dysfunction.

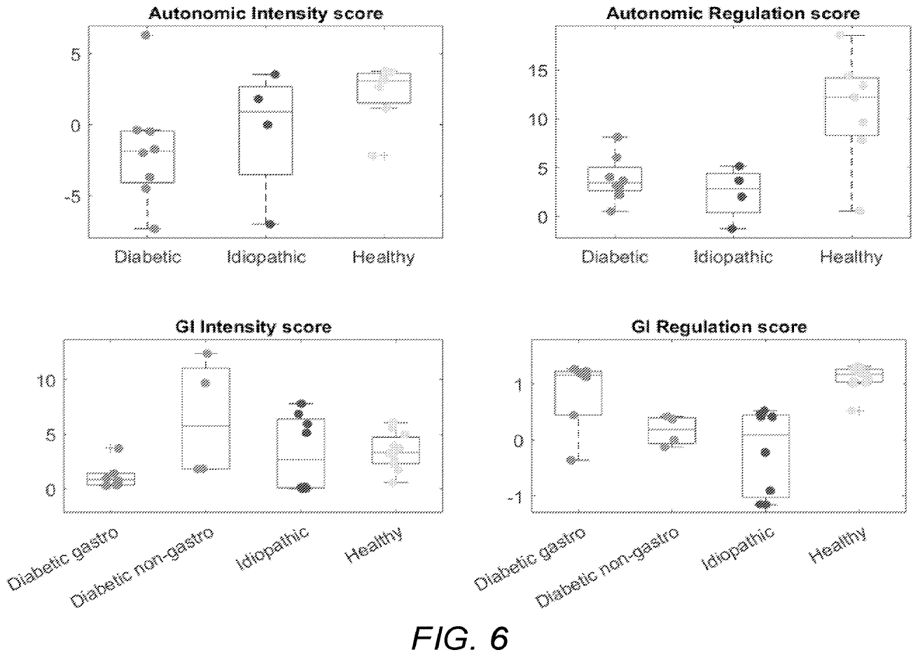

FIG. 6 illustrates box plots of the various dysfunction scores. The autonomic intensity score is highest for the healthy controls as a group and lowest for the diabetics, with the idiopathic subgroup in between. The autonomic regulation score is highest for the healthy controls, lowest for the idiopathic patients, and only slightly higher for the diabetic patients. FIG. 6 quantifies the observation that diabetics tend to have decreased intensity of activity and dysregulation, while idiopathic gastroparesis patients tend to have more dysregulation than decreased intensity. Similarly, the gastric motility intensity score is higher for all three groups except for the diabetic gastroparesis group. The gastric motility regulation score is higher for the healthy controls and diabetic gastroparesis patients than for the diabetic non-gastroparesis and idiopathic gastroparesis patients. The two gastric neuromuscular scores can separate diabetic gastroparesis from diabetic non-gastroparesis patients, diabetic gastroparesis from idiopathic gastroparesis, and all the patients from controls. Combining the autonomic and gastric motility scores can give even more nuanced information to separate intersecting autonomic and GI phenotypes.

### Hardware Implementation

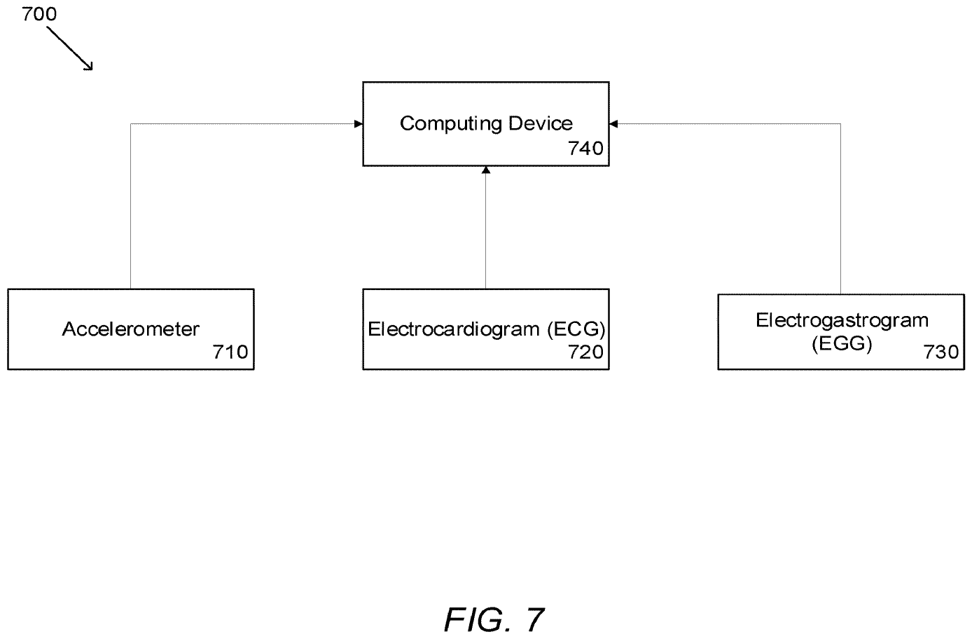

FIG. 7 illustrates a hardware architecture of a system to identify gastric dysfunction in accordance with an embodiment of the invention. In numerous embodiments, system 700 includes accelerometer 710, ECG 720, and EGG 730. The accelerometer may be a triaxial accelerometer that can be used to identify when a user of the system has gone to sleep and woken up. The ECG and EGG can be used to monitor the user's autonomic and gastric myoelectric activities. In several embodiments, the accelerometer, ECG, and EGG are connected to a computing device 740, where the computing device can further analyze and classify the user based on monitored autonomic and gastric myoelectric activities.

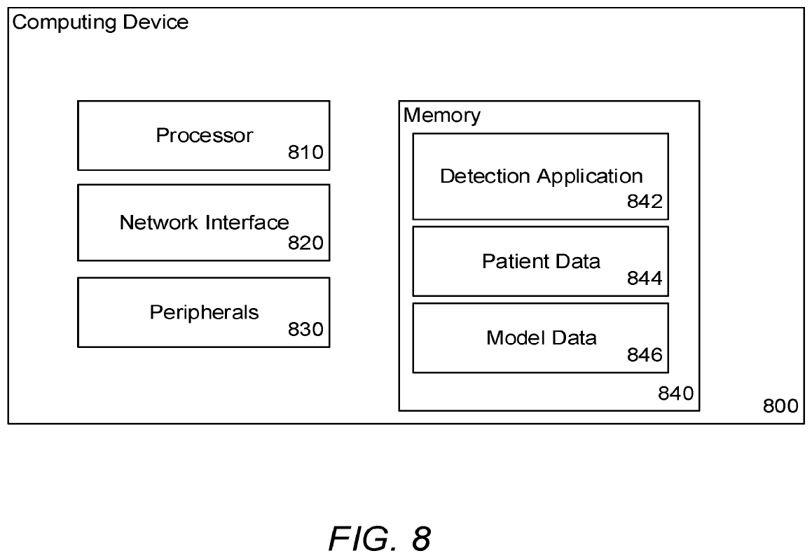

Processes that provide the methods and systems for determining GI phenotype in accordance with some embodiments are executed by a computing device or computing system, such as a desktop computer, tablet, mobile device, laptop computer, notebook computer, server system, and/or any other device capable of performing one or more features, functions, methods, and/or steps as described herein. FIG. 8 illustrates a computing device that can be utilized to perform classification and detection of gastroparesis in accordance with an embodiment of the invention. Computing device 800 includes a processor 810. Processor 810 may direct the detection application 842 to perform classification and detection of gastroparesis in a patient based on patient data 844. In many embodiments, processor 810 can include a processor, a microprocessor, a controller, or a combination of processors, microprocessor, and/or controllers that performs instructions stored in the memory 840 to classify and detect gastroparesis. Processor instructions can configure the processor 810 to perform processes in accordance with certain embodiments of the invention. In various embodiments, processor instructions can be stored on a non-transitory machine readable medium. Computing device 800 further includes a network interface 820 that can receive patient data from external sources, including the accelerometer, ECG, and EEG. In numerous embodiments, computing device 800 includes peripherals 830. Peripherals 830 may include triaxial accelerometers, ECGs, and EGGs that are implemented onboard the computing device such that the computing device is a wearable device capable of performing ambulatory monitoring of autonomic and gastric myoelectric activities of the wearer. Computing device 800 may further include a memory 840 to store classifier data under model data 846. In some embodiments, computing device 800 can be used in the hardware architecture illustrated in FIG. 7.

Although a specific example of a computing device is illustrated in this figure, any of a variety of computing devices can be utilized to classify and detect gastroparesis similar to those described herein as appropriate to the requirements of specific applications in accordance with embodiments of the invention.

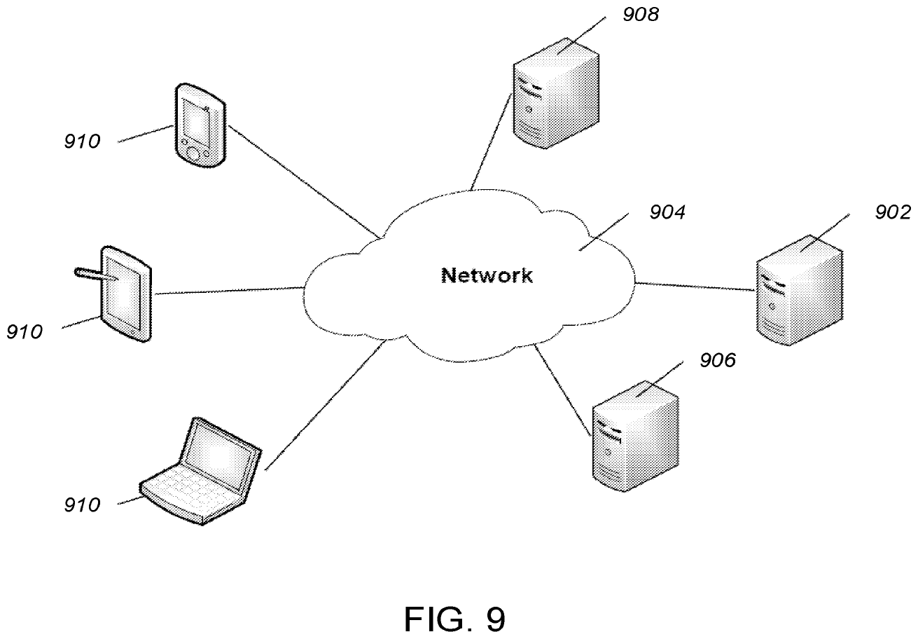

FIG. 9 illustrates a network architecture for classifying and detecting gastroparesis in accordance with an embodiment of the invention. Such embodiments may be useful where computing power is not possible at a local level, and a central computing device (e.g., server) performs one or more features, functions, methods, and/or steps described herein. In such embodiments, a computing device 902 (e.g., server) is connected to a network 904 (wired and/or wireless), where it can receive inputs from one or more computing devices, including data from a records database or repository 906, data provided from a laboratory computing device 908, and/or any other relevant information from one or more other remote devices 910. Once computing device 902 performs one or more features, functions, methods, and/or steps described herein, any outputs can be transmitted to one or more computing devices 906, 908, and 910 for entering into records.

In accordance with still other embodiments, the instructions for the processes can be stored in any of a variety of non-transitory computer readable media appropriate to a specific application.

### Doctrine of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method for determining a gastrointestinal (GI) phenotype, comprising:
obtaining an electrophysiological trace for an individual;
extracting a plurality of autonomic features and a plurality of gastric motility features from the electrophysiological trace;
generating at least one autonomic dysfunction score based on the extracted plurality of autonomic features;
generating at least one gastric dysfunction score based on the extracted plurality of gastric motility features; and
determining a GI phenotype based on the extracted autonomic and gastric motility features and the generated autonomic and gastric dysfunction scores.

2. The method of claim 1, wherein the electrophysiological trace includes electro cardiogram (ECG) and electrogastrogram (EGG) data.

3. The method of claims 1 or 2, wherein the extracted plurality of features comprises instantaneous low frequency power (LF), high frequency power (HF), sympathovagal balance (LF/HF), normalized LF (LFnu), and total autonomic modulation (Totpow).

4. The method of any of claims 1-3, wherein the phenotype is selected from: healthy, diabetic gastropareses, diabetic non-gastroparesis, and idiopathic gastroparesis.

5. The method of any of claims 1-4, wherein extracting the plurality of features comprises:
isolating gastric myoelectric information;
isolating autonomic information; and
identifying sleep and wake times.

6. The method of any of claims 1-5, where in the sleep and wake times are identified using accelerometer data.

7. The method of any of claims 1-6, wherein the electrophysiological trace is obtained for at least 24 hours.

8. The method of any of claims 1-7, wherein the electrophysiological trace is obtained via an ambulatory EGG.

9. The method of any of claims 1-8, wherein the at least one dysfunction score is generated using functional principal components analysis (fPCA).

10. The method of any of claims 1-9, wherein the extracted plurality of features is used to train a classifier to classify GI phenotypes.

11. The method of any of claims 1-10, wherein the classifier comprises multinomial regression, K-nearest neighbor (KNN) with one neighbor, and support vector machine (SVM) with a linear kernel.

12. The method of any of claims 1-11, further comprising generating a treatment plan based on the determined GI phenotype.

13. The method of any of claims 1-12, further comprising determining an autonomic phenotype based on the extracted plurality of features and the at least one dysfunction score.

14. A computer-readable medium comprising instructions which, when executed by a computer, cause the computer to carry out the method of claims 1-13.

15. A system for determining a gastrointestinal (GI) phenotype, the system comprising:
an accelerometer;
an ECG;
an EGG;
a memory; and
a processor comprising a set of one or more processors and a memory containing an identification application, wherein the identification application configures the set of processors to carry out the method of claims 1-13.

16. A method for determining a gastrointestinal (GI) phenotype, comprising:
obtaining an electrophysiological trace for an individual;
extracting a plurality of autonomic features and a plurality of gastric motility features from the electrophysiological trace; and
determining a GI phenotype based on the extracted autonomic and gastric motility features.
